(19) United States
(12) Patent Application Publication
(10) Pub. No.: US 2007/0134196 A1
(43) Pub. Date: Jun. 14, 2007

(54) Personal portable content player with aroma function and method thereof

(76) Inventors: Se Ho Park, Daejeon-city (KR); Moon Youn Jung, Daejeon-city (KR); Hyeon-Bong Pyo, Daejeon-cityq (KR); Chang Auck Choi, Daejeon-city (KR)

Correspondence Address: LADAS & PARRY LLP, 224 SOUTH MICHIGAN AVENUE, SUITE 1600, CHICAGO, IL, 60604, US

(21) Appl. No.: 11/633,173

(22) Filed: Dec. 4, 2006

(30) Foreign Application Priority Data
Dec. 8, 2005 (KR) 10-2005-0120099
Mar. 16, 2006 (KR) 10-2006-0024270

(51) Int. Cl. A61L 9/00; G11B 17/04

(52) U.S. Cl. 424/76.2; 369/200

(57) ABSTRACT

A personal portable content player with an aroma function and a content reproducing method thereof are provided. The personal portable content player includes a storage unit storing content, an aroma chip, which is inserted into a predetermined position of the personal portable content player and diffuses an aromatic material when the content is reproduced, an aromatic material number detector detecting the number of the aromatic material included in the content, and a control unit controlling the aroma chip to diffuse the aromatic material corresponding to the aromatic material number detected by the aromatic material number detector. Accordingly, it is possible to provide a personal portable content player with an aroma function and a content reproducing method thereof capable of providing an aroma function in addition to video and audio functions.

### CROSS-REFERENCE TO RELATED PATENT APPLICATION

This application claims the benefit of Korean Patent Application Nos. 10-2005-0120099, filed on Dec. 8, 2005 and 10-2006-0024270, filed on Mar. 16, 2006 in the Korean Intellectual Property Office, the disclosures of which are incorporated herein in their entirety by reference.

### BACKGROUND OF THE INVENTION

1. Field of the Invention

The present invention relates to a personal portable content player and a method thereof, and more particularly, to a personal portable content player having an aroma function in addition to conventional audio and video functions and a method thereof.

2. Description of the Related Art

At present, examples of personal portable content player being produced and sold are laptop computers, palmtop computers, personal digital assistants (PDAs), portable media players (PMPs), MP3 players, and portable media recorders (PMCs). The various personal portable content players occupy a large portion of the IT industry market.

In general, conventional personal portable content players provide audio and video functions for pictures, motion pictures, and sound sources (music). However, they cannot provide an aroma function.

### SUMMARY OF THE INVENTION

The present invention provides a personal portable content player which includes an aroma chip at a predetermined position and provides an aroma function in addition to conventional audio and video functions and a method thereof.

According to an aspect of the present invention, there is provided a personal portable content player with an aroma function including: a storage unit storing contents; and an aroma chip which is inserted in a predetermined position in the personal portable content player and diffuses an aromatic material when a content in the storage unit is reproduced.

In the above aspect of the present invention, the personal portable content player may further include: an aromatic material number detector detecting the number of the aromatic material included in the content; and a control unit controlling the aroma chip to diffuse the aromatic material corresponding to the aromatic material number detected by the aroma material number detector.

According to another aspect of the present invention, there is provided a content reproducing method using a personal portable content player with an aroma function, the method including: (a) detecting an aromatic material number included in a content to be reproduced among the contents stored in the personal portable content player; (b) detecting a chamber in an aroma chip, which contains an aromatic material corresponding to the detected aromatic material number; and (c) controlling the aroma chip to diffuse the aromatic material contained in the chamber by heating the chamber detected in (b).

### BRIEF DESCRIPTION OF THE DRAWINGS

The above and other features and advantages of the present invention will become more apparent by describing in detail exemplary embodiments thereof with reference to the attached drawings in which:

FIG. 1 is a block diagram of a personal portable content player with an aroma function according to an embodiment of the present invention;

FIGS. 2A to 2C show detailed examples of an aroma chip of FIG. 1; and

FIG. 3 is a flowchart of a content reproducing method using a personal portable content player with an aroma function according to another embodiment of the present invention.

### DETAILED DESCRIPTION OF THE INVENTION

A personal portable content player having an aroma function and method thereof according to the present invention will be described in detail with reference to the accompanying drawings, in which exemplary embodiments of the invention are shown.

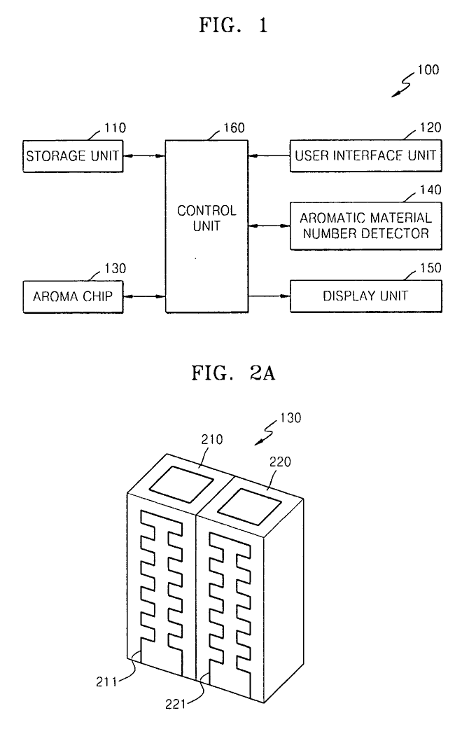

FIG. 1 is a block diagram of a personal portable content player with an aroma function according to an embodiment of the present invention. Referring to FIG. 1, the personal portable content player 100 includes a storage unit 110, a user interface unit 120, an aroma chip 130, an aromatic material number detector 140, a display unit 150, and a control unit 160.

Various contents to be reproduced are stored in the storage unit 110. In addition, the storage unit 110 includes an Electrically-Erasable Programmable Read-Only Memory (EEPROM) for storing programs and initial service data for operating the personal portable content player 100 and a Random-Access Memory (RAM) for temporarily storing data generated during an operation of the personal portable content player 100.

Here, each content item stored in the storage unit 110 includes an aromatic material number. Specifically, when the aroma chip 130 in the personal portable content player 100 is standardized, users sharing the same contents can smell the same aroma using the same aromatic material by assigning the aromatic material number to the aromatic material in the aroma chip 130 according to contents.

The user interface unit 120 performs an input/output interface for receiving information from a user, about the content to be reproduced from among contents stored in the storage unit 110.

Furthermore, according to another embodiment of the present invention, the aromatic material number to be used together with the content information, in addition to the information about the content to be reproduced, can be input through the user interface unit 120. The user interface unit 120 transmits the aromatic material number together with the content information to the control unit 160.

More specifically, the user interface unit 120 includes an infrared (IR) receiver receiving a command input from a keypad or IR remote. The IR remote may be a remote controller (REMOCN) or a wireless mouse such as an air mouse.

The aroma chip 130 that is inserted in a predetermined position in the personal portable content player provides the aroma function by using the aromatic material in the aroma chip 130 when the content is reproduced.

A chamber in the aroma chip 130 contains the aromatic material. The aroma chip 130 is constructed so that a resistor heater for heating and diffusing the aromatic material is adhered to the chamber in the aroma chip 130.

In addition, there are a plurality of chambers included inside the aroma chip 130. Each chamber includes a different aromatic material. The aroma chip 130 is constructed so that each resistor heater for heating and diffusing the aroma material is adhered to each chamber.

The resistor heater adhered to the chamber in the aroma chip 130 is heated using its own battery or a battery of the personal portable content player 100.

The aromatic material number detector 140 detects the aromatic material number included in the content to be reproduced using the user interface unit 120.

Furthermore, when the aromatic material number, in addition to the content information, is directly input by the user through the user interface unit 120, the aromatic material number detector 140 is not used.

The control unit 160 controls the aroma chip 130 to diffuse the aromatic material corresponding to the aromatic material number detected by the aromatic material number detector 140. More specifically, when the aroma chip 130 includes a plurality of chambers, the control unit 160 controls the aroma chip 130 to diffuse the corresponding aromatic material by detecting the chamber corresponding to the aromatic material number and heating the resistor heater of the detected chamber.

Furthermore, when the aromatic material number, in addition to the content information, is directly input by the user through the user interface unit 120, the control unit 160 controls the aroma chip 130 to diffuse the aromatic material corresponding to the aromatic material number received from the user interface unit 120.

The display unit 150 displays the title of the content that is reproduced by the personal portable content player 100 under the control of the control unit 160 so as to be recognizable to the user.

Examples of the personal portable content player of FIG. 1 are a laptop computer, a palmtop computer, a personal digital assistant (PDA), a portable media player (PMP), an MP3 player, and a portable media recorder (PMC).

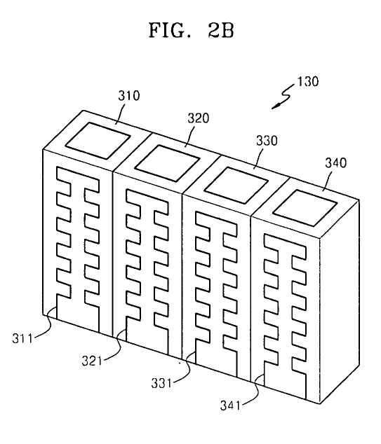

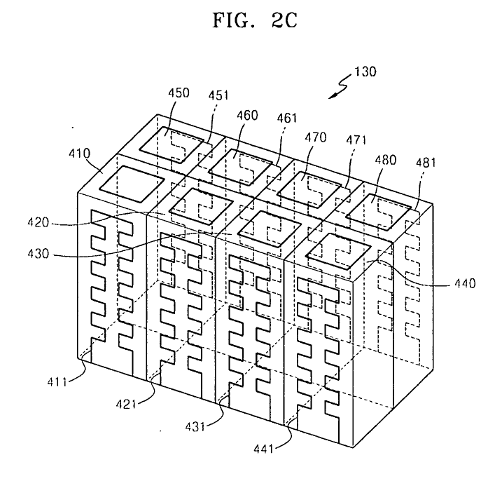

FIGS. 2A to 2C show detailed examples of an aroma chip of FIG. 1.

Referring to FIG. 2A, the aroma chip 130 includes first and second chambers 210 and 220. First and second resistor heaters 211 and 212 are adhered to the first and second chambers 210 and 220, respectively.

Referring to FIG. 2B, the aroma chip 130 includes first to fourth chambers 310 to 340. First to fourth resistor heaters 311 to 341 are adhered to the first to fourth chambers 310 to 340, respectively.

Referring to FIG. 2C, the aroma chip 130 includes first to eighth chambers 410 to 480. First to eighth resistor heaters 411 to 481 are adhered to the first to eighth chambers 410 to 480, respectively.

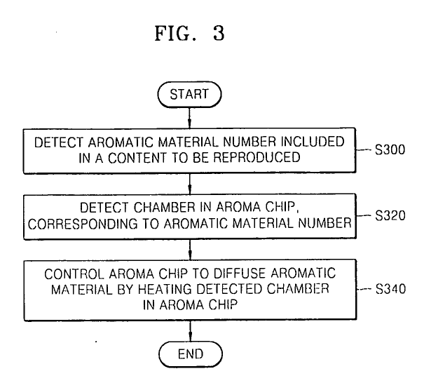

FIG. 3 is a flowchart of a content reproducing method using a personal portable content player with an aroma function according to another embodiment of the present invention. Referring to FIG. 3, first, the aromatic material number included in the content to be reproduced among the contents stored in the personal portable content player is detected (operation S300).

Next, the chamber in the aroma chip which contains the aromatic material corresponding to the aromatic material number detected in operation S300 is detected (S320).

This chamber in the aroma chip contains the aromatic material, and the resistor heater for heating and diffusing the aromatic material is adhered to the chamber.

In addition, the aroma chip 130 includes a plurality of chambers inside. The plurality of chambers contain different aromatic materials, respectively. The aroma chip 130 is constructed so that each resistor heater for heating and diffusing the aromatic material is adhered to each chamber.

Next, the aroma chip is controlled to diffuse the aromatic material contained in the chamber by heating the chamber detected in operation S320 (operation S340).

In detail, in operation S340, the corresponding resistor heater among the resistor heaters which are adhered to the chamber in the aroma chip and are used for heating and diffusing the aromatic materials in the chambers is controlled to be heated.

Examples of the personal portable content player of FIG. 3 are a laptop computer, a palmtop computer, a PDA, a PMP, an MP3 player, and a PMC.

A part which is not described in FIG. 3 may be referred to in the descriptions of FIGS. 1 and 2.

The invention can also be embodied as computer readable codes on a computer readable recording medium. The computer readable recording medium is any data storage device that can store data which can be thereafter read by a computer system. Examples of the computer readable recording medium include read-only memory (ROM), random-access memory (RAM), CD-ROMs, magnetic tapes, floppy disks, optical data storage devices, and carrier waves (such as data transmission through the Internet). The computer readable recording medium can also be distributed over network coupled computer systems so that the computer readable code is stored and executed in a distributed fashion.

The personal portable content player and the method thereof according to the present invention have the following advantages.

The personal portable content player basically provides pictures, motion pictures, and sound sources (music), and is also used for learning languages. The personal portable content player can help to improve recognition, memorization, and learning by additionally providing an aroma function.

When a user watches, or listens to, the content reproduced by the personal portable content player, the user may become tired. The personal portable content player can help the user to overcome fatigue and to ease body and mind by providing the aroma function.

In addition, when the aroma chip in the personal portable content player is standardized, users sharing the same contents can smell the same aroma using the same aromatic material by assigning the aromatic material number to the aroma material in the aroma chip according to contents.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A personal portable content player with an aroma function comprising:
a storage unit storing contents; and
an aroma chip, which is inserted in a predetermined position in the personal portable content player and diffuses an aromatic material when a content in the storage unit is reproduced.

2. The personal portable content player of claim 1, further comprising:
an aromatic material number detector detecting an aromatic material number included in the content; and
a control unit controlling the aroma chip to diffuse the aromatic material corresponding to the aromatic material number detected by the aromatic material number detector.

3. The personal portable content player of claim 1, wherein a chamber in the aroma chip contains the aroma material.

4. The personal portable content player of claim 3, wherein a resistor heater for heating and diffusing the aromatic material is adhered to the chamber in the aroma chip.

5. The personal portable content player of claim 1, wherein a resistor heater for heating and diffusing the aromatic material is adhered to a chamber in the aroma chip.

6. The personal portable content player of claim 1, wherein the aroma chip includes a plurality of chambers, and the plurality of chambers include different aromatic materials, respectively.

7. The personal portable content player of claim 6, wherein a resistor heater for heating and diffusing the aromatic material is adhered to each chamber.

8. The personal portable content player of claim 7, further comprising:
an aromatic material number detector detecting an aromatic material number included in the content; and
a control unit controlling the aroma chip to diffuse the aromatic material corresponding to the aromatic material number detected by the aromatic material number detector.

9. The personal portable content player of claim 6, further comprising:
an aromatic material number detector detecting an aromatic material number included in the content; and
a control unit controlling the aroma chip to diffuse the aromatic material corresponding to the aromatic material number detected by the aromatic material number detector.

10. The personal portable content player of claim 1, further comprising:
a user interface unit receiving an aromatic material number to be used together with an information of the contecnt; and
a control unit controlling the aroma chip to diffuse the aromatic material corresponding to the aromatic material number received from the user interface unit.

11. A content reproducing method using a personal portable content player with an aroma function, the method comprising:
(a) detecting an aromatic material number included in a content to be reproduced from among the contents stored in the personal portable content player;
(b) detecting a chamber in an aroma chip which contains an aromatic material corresponding to the detected aromatic material number; and
(c) controlling the aroma chip to diffuse the aromatic material contained in the chamber by heating the chamber detected in (b).

12. The method of claim 11,
wherein a resistor heater for heating and diffusing the aromatic material is adhered to the chamber in the aroma chip, and
wherein (c) is performed by using the resistor heater.
